(19) United States
(12) Patent Application Publication
(10) Pub. No.: US 2023/0058735 A1
(43) Pub. Date: Feb. 23, 2023

(54) ACCELERATOR AND PARTICLE THERAPY SYSTEM

(71) Applicant: HITACHI, LTD., Tokyo (JP)

(72) Inventors: Yuto NAKASHIMA, Tokyo (JP); Takamichi AOKI, Tokyo (JP)

(21) Appl. No.: 17/795,611

(22) PCT Filed: Aug. 24, 2020
(86) PCT No.: PCT/JP2020/031754

§ 371 Date: Jul. 27, 2022

(30) Foreign Application Priority Data
Mar. 25, 2020 (JP) 2020-053547

(51) Int. Cl. H05H 7/04; A61N 5/10

(52) U.S. Cl. CPC H05H 7/04; A61N 5/1048; A61N 2005/1074; H05H 2007/043

(57) ABSTRACT

An object of the present invention is to prevent disappearance of ions supplied to an accelerator. An eccentric trajectory type accelerator 1 includes a laser source 12 and a target 20 that emits ions by being irradiated with a laser beam emitted from the laser source 12. The eccentric trajectory type accelerator 1 includes a container 10 that forms a columnar space therein, an acceleration electrode structure that accelerates ions in a circumferential direction of the columnar space, and a main coil 38 that generates a magnetic field in an axial direction of the columnar space, and accelerates the ions emitted from the target 20. The target 20 is disposed at a position away from a central axis of the columnar space.

### TECHNICAL FIELD

The present invention relates to an accelerator and a particle therapy system, and particularly to a technique for supplying ions to an accelerator.

### BACKGROUND ART

Particle therapy in which a target volume is irradiated with a particle beam is widely performed. In general, a particle therapy system including an accelerator is used in particle therapy. Ions such as carbon ions, helium ions, and protons are injected into the accelerator, and the ions are accelerated until the ions have energy necessary for therapy. The particle therapy system irradiates the target volume with a charged particle beam by the ions accelerated by the accelerator. In the particle therapy system, the energy and spatial spreading of the charged particle beam is adjusted in accordance with a position and a shape of the target volume.

The accelerator includes a cyclotron that accelerates ions injected into a magnetic field by a radiofrequency electric field and causes the ions to travel in a container. In the cyclotron, the ions travel while increasing a trajectory radius together with acceleration. The energy of the ions increases as the trajectory radius increases, and the ions are extracted when the energy reaches the maximum energy. In the cyclotron, since the ions are extracted when the energy reaches the maximum energy, it is difficult to control the energy of the charged particle beam.

The accelerator also includes a synchrocyclotron described in PTL 1 and NPL 1 below. Unlike the cyclotron in which the ions are accelerated by the radiofrequency electric field having a constant frequency, a frequency of the radiofrequency electric field is modulated according to a change in motion period caused by a mass change of the ions in the synchrocyclotron. Due to this frequency modulation, the ions are accelerated when the ions pass through a region where a radiofrequency electric field is generated. In the synchrocyclotron, it is difficult to control the energy of the charged particle beam to be extracted for the same reason as the cyclotron.

PTL 2 and PTL 3 below describe an eccentric trajectory type accelerator in which energy of extracted ions can be controlled. In a general cyclotron, an ion injection port is provided at a center of an upper surface of a container in which the ions travels, and an ion extraction port is provided on a side surface of the container.

On the other hand, in the eccentric trajectory type accelerator, the ion injection port is provided at a position shifted toward the beam extraction port side from the center of the upper surface of the container. As a result, a plurality of beam closed trajectories around which ions having different energies travel around become dense on the beam extraction port side. Accordingly, in the vicinity of the beam extraction port, it is easy to separate the ions having different energies from the beam closed trajectories, and charged particle beams having different energies are efficiently acquired. Note that, NPL 2 describes a technique of injecting ions from an outside of an accelerator.

### CITATION LIST

### Patent Literature

PTL 1: JP2013-541170A

PTL 2: JP2019-96404A

PTL 3: WO2016/092621A

### Non-Patent Literature

NPL 1: W. Kleeven, “The IBA Superconducting Synchrocyclotron Project S2C2”, Proceedings of Cyclotrons 2013

NPL 2: P. Mandrillon, Injection into cyclotrons, CAS, CERN 96-02, (1996), p. 153.

### SUMMARY OF INVENTION

### Technical Problem

A tubular through-hole extending from the upper surface to the internal space is formed in the container constituting the eccentric trajectory type accelerator, and an opening of the through-hole in the upper surface of the container is the ion injection port. A coil is provided in the container, and the coil generates a magnetic field in the container to deflect the ions. The magnetic field generated by the coil passes through the through-hole. Thus, when the ions are injected into the through-hole, the ions may be deflected by the magnetic field passing through the through-hole, and the ions may collide with an inner wall of the through-hole and may disappear.

An object of the present invention is to prevent disappearance of ions supplied to an eccentric trajectory type accelerator.

### Solution to Problem

The present invention includes a laser source and a target that emits ions by being irradiated with a laser beam emitted from the laser source, and accelerates the ions emitted from the target.

### Advantageous Effects of Invention

According to the present invention, the disappearance of the ions supplied to the accelerator can be prevented.

### BRIEF DESCRIPTION OF DRAWINGS

FIG. 1 is a perspective view of an eccentric trajectory type accelerator.

FIG. 2 is a diagram illustrating a portion on a lower side of the eccentric trajectory type accelerator as viewed from above.

FIG. 3 is a cross-sectional view of the eccentric trajectory type accelerator.

FIG. 4 is a diagram schematically illustrating structures of a dee electrode and a dummy dee electrode.

FIG. 5 is a perspective view of an ion generator.

FIG. 6 is a cross-sectional view of the ion generator.

FIG. 7 is a diagram illustrating a position where the ion generator is provided.

FIG. 8 is a diagram illustrating the portion on the lower side of the eccentric trajectory type accelerator as viewed from above.

FIG. 9 is a cross-sectional view of the eccentric trajectory type accelerator.

FIG. 10 is a diagram illustrating a particle therapy system.

### DESCRIPTION OF EMBODIMENTS

An embodiment of the present invention will be described with reference to the drawings. The same components illustrated in the plurality of drawings are denoted by the same reference signs, and the description thereof is simplified. Terms representing shapes such as “columns” in the present specification do not indicate only geometrically strictly defined shapes. The terms representing the shapes in the present specification also indicate shapes to which deformation is applied within a range in which a function of the component can be secured.

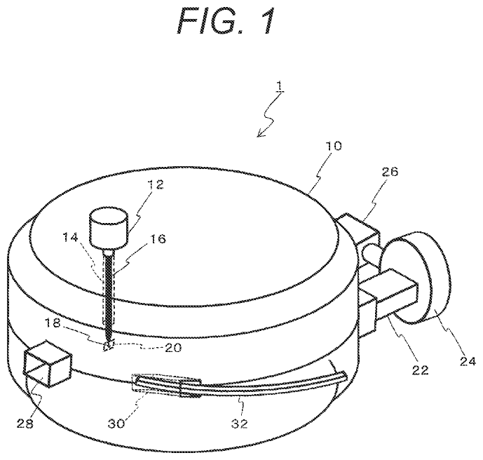

FIG. 1 is a perspective view of an eccentric trajectory type accelerator 1. An outer shell of the eccentric trajectory type accelerator 1 is formed by a container 10 that can be divided into upper and lower portions. The container 10 forms a column-shaped space (columnar space) therein. Although an outer surface of the container 10 in the present embodiment has a column shape, the shape formed by the outer surface of the container 10 is not necessarily a column shape.

An upper portion which is a portion on an upper side of the container 10 and a lower portion which is a portion on a lower side of the container have container shapes in which a cylindrical upper end and a cylindrical lower end are closed, respectively. The upper portion and the lower portion are joined with openings facing each other, and an internal space is evacuated. As will be described later, each of the upper portion and the lower portion is a magnet that generates a magnetic field in the internal space of the container 10.

A laser source 12 is provided on an upper surface of the upper portion. The laser source 12 may be, for example, a carbon dioxide laser, a helium neon laser, a YAG laser, a titanium sapphire laser, or the like. A laser injection through-hole 14 extending from an outside of the container 10 to the internal space is provided at a position where the laser source 12 is provided in the upper portion. A target 20 is disposed at an ion injection point 18 is present at a position immediately below the laser injection through-hole 14.

The target 20 is made of a material that generates ions by being irradiated with a laser beam. The target 20 may be made of a material containing carbon. The target 20 may be formed by using, for example, a metal plate on which carbon is deposited.

The laser source 12 generates a laser beam 16 toward the laser injection through-hole 14. The target 20 is irradiated with the laser beam 16 via the laser injection through-hole 14. When the intensity of the laser beam 16 focused on the target 20 is sufficient to ionize atoms constituting the target 20, plasma is generated. As will be described later, the eccentric trajectory type accelerator 1 includes an ion generator including the target 20. The ion generator supplies ions to the ion injection point 18 by separating electrons and ions from the plasma.

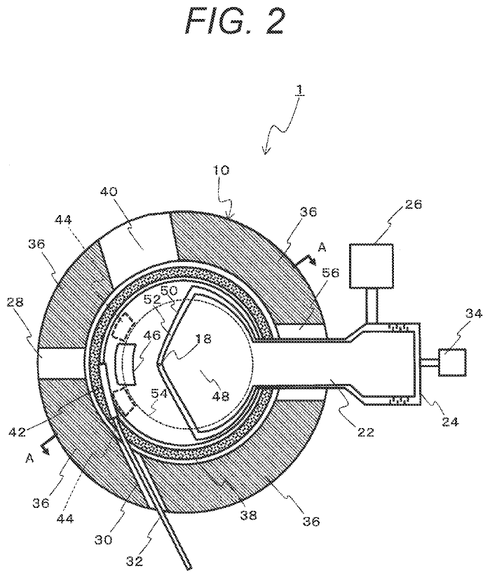

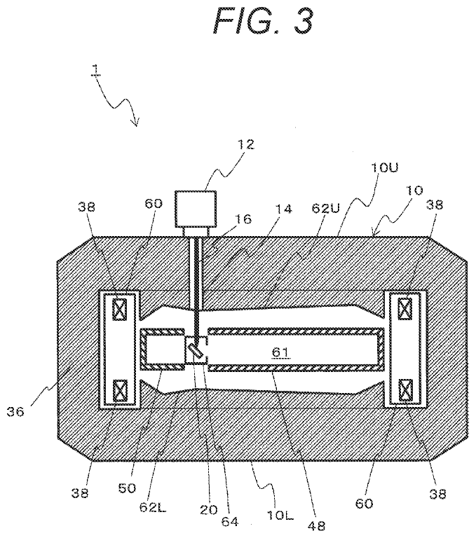

FIG. 2 is a diagram illustrating a portion on a lower side of the eccentric trajectory type accelerator 1 in a state of being divided into upper and lower portions as viewed from above. FIG. 3 is a cross-sectional view of the eccentric trajectory type accelerator 1 taken along line AA of FIG. 2. As illustrated in FIG. 3, the container 10 includes a disk-shaped top plate 10U, a disk-shaped bottom plate 10L, an upper magnetic pole 62U, a lower magnetic pole 62L, and a yoke 36. The magnetic pole 62U is a portion protruding downward from the top plate 10U. The magnetic pole 62 L is a portion protruding upward from the bottom plate 10L. The yoke 36 is an annular wall extending from a periphery of the bottom plate 10L to a periphery of the top plate 10U.

Annular main coils 38 are arranged between the upper magnetic pole 62 U and the yoke 36 and between the lower magnetic pole 62 L and the yoke 36, respectively. The upper and lower main coils 38 travel around an upper end portion and a lower end portion of the columnar space inside the container 10. Each of the main coils 38 is a superconducting coil. A cryostat 60 is disposed around each main coil 38, and the cryostat 60 cools each main coil 38. The upper main coil 38 and the upper portion of the container 10 constitute an upper magnet, and the lower main coil 38 and the lower portion of the container 10 constitute a lower magnet.

The upper magnetic pole 62U and the lower magnetic pole 62L face each other, and a region sandwiched between the magnetic poles 62U and 62L includes an ion passage region 61 through which ions pass while traveling. Each of the main coils 38 generates a magnetic field in an axial direction of the columnar space, and generates a magnetic field passing through the magnetic poles 62U and 62L and the ion passage region 61 in a vertical direction.

As illustrated in FIG. 2, a beam through-hole 30 for extracting an accelerated charged particle beam is provided in the yoke 36. A conductive wire through-hole 28 for extracting various conductive wires inside the container 10, a vacuuming extraction through-hole 40, and a radiofrequency system through-hole 56 in which a radiofrequency acceleration cavity 22 is disposed in the yoke 36.

The eccentric trajectory type accelerator 1 includes an acceleration radiofrequency power supply 26, the radiofrequency acceleration cavity 22, a dee electrode 48, and a dummy dee electrode 50. The acceleration radiofrequency power supply 26 excites a radiofrequency magnetic field in the radiofrequency acceleration cavity 22. The radiofrequency acceleration cavity 22 generates a radiofrequency electric field for accelerating ions in the dee electrode 48. The radiofrequency acceleration cavity 22 includes a variable capacitor 24 for modulating a frequency of the radiofrequency electric field by changing its own resonance frequency, and a motor 34 for changing an electrostatic capacitance of the variable capacitor 24.

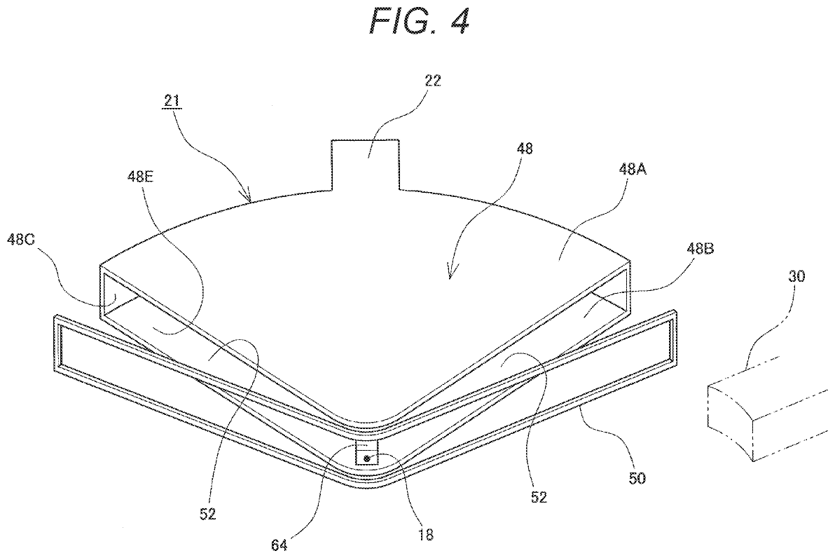

FIG. 4 schematically illustrates structures of the dee electrode 48 and the dummy dee electrode 50. The dee electrode 48 and the dummy dee electrode 50 constitute an acceleration electrode structure that accelerates ions in a circumferential direction of the columnar space. The dee electrode 48 has a structure in which a cavity upper wall 48A and a cavity lower wall 48B having a spread-out shape (a shape approximating a fan shape) face each other and an outer periphery of the cavity upper wall 48A and an outer periphery of the cavity lower wall 48B are connected by a cavity sidewall 48C. However, an opening is provided in a central portion of the cavity sidewall 48C, and a tubular radiofrequency acceleration cavity 22 extending in a direction away from the cavity upper wall 48A and the cavity lower wall 48B is formed in the opening.

The cavity upper wall 48A, the cavity lower wall 48B, and the cavity sidewall 48C constitute the dee electrode 48. The dummy dee electrode 50 is formed by an annular conductor surrounding a region facing a dee electrode opening 48E formed by the cavity upper wall 48A, the cavity lower wall 48B, and the cavity sidewall 48C. That is, the dummy dee electrode 50 has an annular conductor facing an edge of the dee electrode opening 48E. An acceleration gap 52 is formed between the dummy dee electrode 50 and the cavity upper wall 48A, the cavity lower wall 48B, and the cavity sidewall 48C.

In this manner, the dee electrode 48 forms a spread-out cavity space surrounded by the cavity upper wall 48A, the cavity lower wall 48B, and the cavity sidewall 48C. The dee electrode 48 has the dee electrode opening 48E that spreads in two different directions as viewed from a base side where the spread-out cavity space spreads. The ion injection point 18 is present between a bent portion of the dummy dee electrode 50 and the dee electrode opening 48E. The ion generator 64 is positioned between the bent portion of the dummy dee electrode 50 and the dee electrode 48, and supplies ions to the ion injection point 18 by irradiating an internal target with a laser beam.

The electrostatic capacitance of the variable capacitor 24 is changed by the rotation of the motor 34, and a resonance frequency of the radiofrequency acceleration cavity 22 is changed (FIG. 2). An acceleration voltage frequency-modulated by a change in the resonance frequency of the radiofrequency acceleration cavity 22 is generated in the acceleration gap 52 between the dee electrode 48 and the dummy dee electrode 50. The ions supplied from the ion generator 64 are accelerated by the acceleration gap, are deflected by a magnetic field in the vertical direction, and travel in the ion passage region.

The ions pass through the spread-out cavity space sandwiched between the cavity upper wall 48A and the cavity lower wall 48B in an arc, pass through the acceleration gap 52 on a left side, and pass under a left side of the dummy dee electrode 50. The ions further pass through a near side of the dummy dee electrode 50 in an arc, pass through a right side of the dummy dee electrode 50, pass through the acceleration gap 52 on a right side, and return to the spread-out cavity space sandwiched between the cavity upper wall 48A and the cavity lower wall 48B. The ions travel around such a trajectory while increasing a trajectory radius, are warped outward by an action of an electric field and a magnetic field to be described later, pass through a high energy beam transport system 32 provided in the beam through-hole 30, and reach an outside of the eccentric trajectory type accelerator 1.

The ions travel around while increasing the trajectory radius so as to approach a maximum energy trajectory 54 indicated by a broken line in FIG. 2. The maximum energy trajectory 54 is a trajectory of the ions reaching a maximum energy. A kick magnetic field generating shim 44 provided at two places for exciting a quadrupole magnetic field and a multipolar magnetic field of six or more poles, and a disturbance electrode 46 to which a radiofrequency voltage is applied are provided in the eccentric trajectory type accelerator 1.

An extraction septum magnet 42 is provided outside the disturbance electrode 46. The kick magnetic field generating shim 44, the disturbance electrode 46, and the extraction septum magnet 42 are used to extract the charged particle beam outward from the eccentric trajectory type accelerator 1.

A radiofrequency voltage is applied to the disturbance electrode 46, and thus, a disturbance electric field is generated from the disturbance electrode 46. The disturbance electric field kicks traveling ions in a direction along a trajectory plane, and causes the ions to depart from the designed trajectory. The ions of which the trajectory departs from a design trajectory pass near the kick magnetic field generating shim 44. The magnetic field generated by the kick magnetic field generating shim 44 restricts a stable region with respect to traveling ions, and introduces ions that exit from the stable region into the extraction septum magnet 42. The ions introduced into the extraction septum magnet 42 pass through the high energy beam transport system 32, and are extracted to the outside of the eccentric trajectory type accelerator 1.

In the eccentric trajectory type accelerator 1, the ion generator 64 including the target 20 is provided at a position away from a central axis of the columnar space. Accordingly, the ion injection point 18 is positioned at a position away from the central axis of the columnar space. A position of the ion injection point 18, that is, a position of the target 20 is a position away from a center of a trajectory of an ion having certain energy.

As a result, a plurality of beam closed trajectories in which ions having different energies travel become denser on a side of the extraction septum magnet 42 than the central axis of the columnar space. Thus, in the vicinity of the extraction septum magnet 42, it becomes easy to separate the ions having different energies from the beam closed trajectories, and charged particle beams having different energies are efficiently acquired.

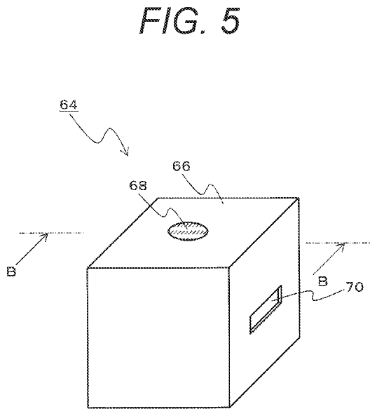

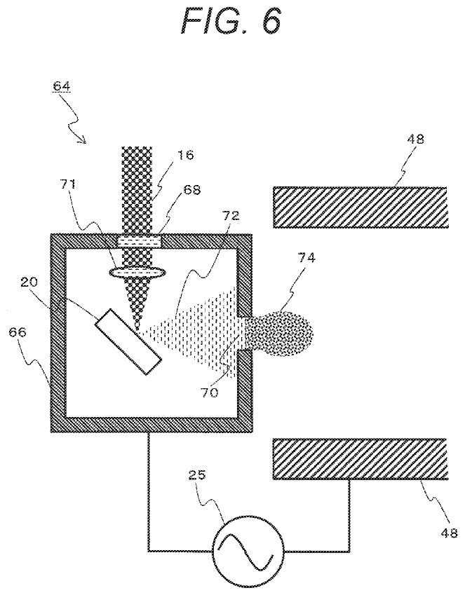

FIG. 5 illustrates a perspective view of the ion generator 64. FIG. 6 is a cross-sectional view of the ion generator 64 taken along line BB illustrated in FIG. 5. The ion generator 64 includes the target 20, a housing 66, and a condenser lens 71. The housing 66 is formed by using a conductor and has a rectangular parallelepiped box shape. The housing 66 accommodates the target 20, and the target 20 is disposed inside the housing 66. An optical vacuum window 68 is provided on an upper surface of the housing 66. The optical vacuum window 68 is made of a material that transmits the laser beam. An ion extraction hole 70 is provided on a side surface of the housing 66. The condenser lens 71 is disposed between the optical vacuum window 68 and the target 20.

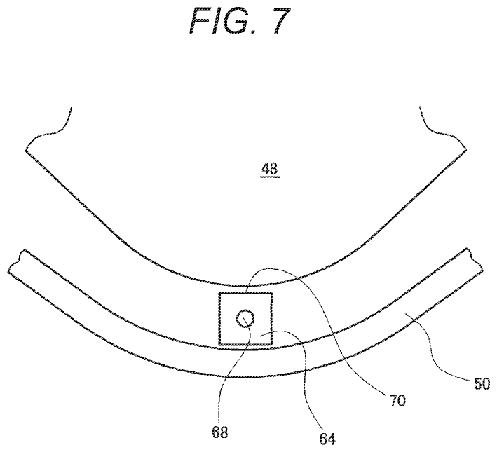

FIG. 7 illustrates a position where the ion generator 64 is provided. The ion generator 64 is disposed between the bent portion of the dummy dee electrode 50 and the dee electrode 48 in a posture in which the optical vacuum window 68 is directed upward and the ion extraction hole 70 is directed toward the spread-out cavity space. Note that, under a condition that the ions are supplied to a region where the ions can be accelerated, a direction of the ion extraction hole 70 may be any direction.

Referring back to FIG. 6, an operation of the ion generator 64 will be described. The laser beam 16 is injected into the target 20 in the housing 66 through the optical vacuum window 68 provided in the housing 66. An AC power supply 25 is connected between the dee electrode 48 and the housing 66. The AC power supply 25 may be the acceleration radiofrequency power supply 26 illustrated in FIG. 2, or may be provided separately from the acceleration radiofrequency power supply 26.

The laser beam 16 injected into the housing 66 is focused on the target 20 by the condenser lens 71. The condenser lens 71 may be a parabolic mirror or an off-axis parabolic mirror. An optical device such as a mirror may be installed to cause the laser beam 16 to pass around.

When the focused laser beam 16 has an intensity sufficient to ionize atoms constituting the target 20, plasma 72 is generated. The focusing intensity may be equal to or greater than 108 W/cm² and equal to or less than 1014 W/cm². The focusing intensity may be changed according to the required amount of charges of ions. The material forming the target 20 may be changed according to the type of ions to be generated. The material of the target 20 may be any of a solid, a liquid, and a gas. When the target 20 is liquid or gas, the amount of charges of ions can be changed by changing a volume.

A plurality of targets made of different materials may be provided in the housing 66. In this case, a plurality of types of ions having different masses or valences emitted from the plurality of targets may be accelerated by the eccentric trajectory type accelerator 1. The valence of the ions to be generated may be switched by changing an irradiation time of the laser beam 16.

A radiofrequency electric field is generated between the housing 66 and the dee electrode 48 by the AC power supply 25. Ions 74 are separated from the plasma 72 by a radiofrequency electric field generated between the housing 66 and the dee electrode 48, and the ions 74 are extracted through the ion extraction hole 70. Note that, the ions 74 may be extracted by using an electrostatic field instead of the radiofrequency electric field. In this case, a DC power supply is connected between the housing 66 and the dee electrode 48, and a DC voltage is applied between the housing 66 and the dee electrode 48.

When the ion generator 64 is irradiated with the laser beam 16 in a shape of a pulse waveform, the amount of charges of ions and a beam time width are controlled by controlling a frequency and a pulse width when the laser beam 16 is generated in a shape of a pulse waveform, for example. Here, the beam time width is defined as a duration in which the charged particle beam is extracted from the eccentric trajectory type accelerator 1. The amount of charges of ions and the beam time width can also be controlled by adjusting a phase of the radiofrequency electric field and an ion generation timing. By such control, a charged particle beam having any amount of charges and any beam time width is extracted from the eccentric trajectory type accelerator 1 at any timing.

Note that, although the eccentric trajectory type accelerator 1 including one laser source 12 has been described above, a plurality of laser sources that irradiate one target with the laser beam may be provided. In this case, a laser injection through-hole may be individually provided for each of the plurality of laser sources. The focusing intensity on the target increases by using the plurality of laser sources.

In the eccentric trajectory type accelerator 1 according to the present embodiment, the target 20 is provided at the ion injection point 18, and the target 20 is irradiated with the laser beam. As a result, the ions are supplied to the ion injection point 18. Accordingly, the disappearance of the ions due to the collision of the ion with an inner wall of the through-hole before the ions are supplied into the accelerator as in the related art in which the ions pass through the through-hole provided in the container is avoided.

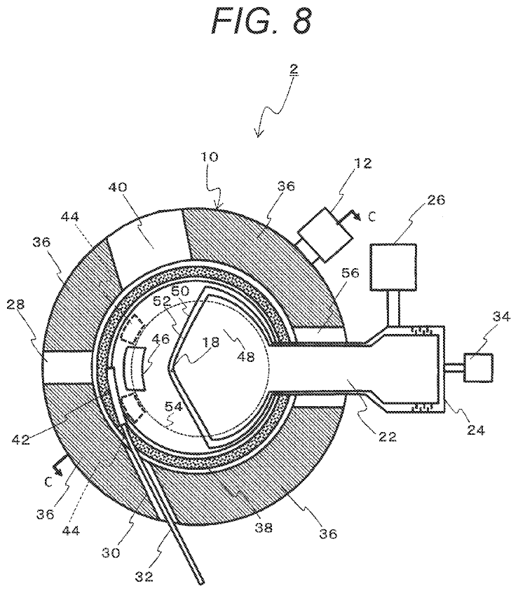

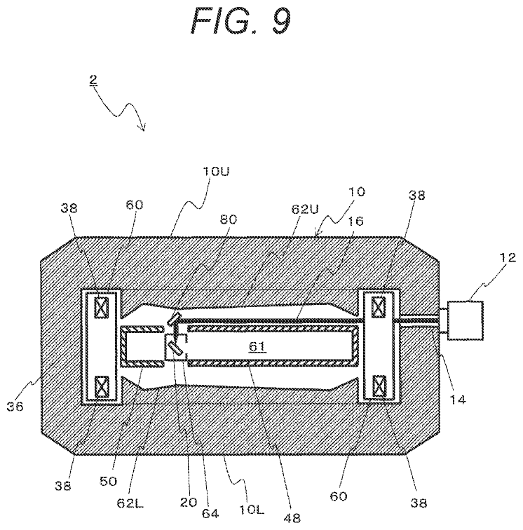

FIGS. 8 and 9 illustrate an eccentric trajectory type accelerator 2 according to a second embodiment. FIG. 8 is a diagram illustrating of a portion on a lower side of the eccentric trajectory type accelerator 2 divided into upper and lower portions. FIG. 9 illustrates a cross-sectional view of the eccentric trajectory type accelerator 2 taken along line CC of FIG. 8.

The eccentric trajectory type accelerator 2 according to the present embodiment is different from the eccentric trajectory type accelerator 2 illustrated in FIGS. 1 to 3 in that the laser source 12 is provided on a side surface of the container 10. As illustrated in FIG. 9, the laser injection through-hole 14 penetrates the yoke 36 on the side surface of the container 10 in a lateral direction. The ion generator 64 present in the internal space of the eccentric trajectory type accelerator 2 is irradiated with the laser beam 16 via the laser injection through-hole 14. That is, the laser beam 16 is reflected by a reflecting member such as an optical mirror 80 and is injected into the ion generator 64.

Here, although the configuration in which the laser source 12 is provided on the side surface of the container 10 has been illustrated, the laser source 12 may be installed on any surface of the container 10. The laser injection through-hole 14 does not necessarily extend in a direction orthogonal to the surfaces of the ceiling part 10U and the yoke 36. An optical path of the laser beam 16 may be formed by using any optical device such as an optical lens or an optical mirror. The optical path of the laser beam 16 may be any optical path from the laser source 12 to the ion generator 64.

In the eccentric trajectory type accelerator 2 according to the second embodiment, the disappearance of the ions due to the collision with a wall surface of the through-hole before the ions are supplied to the ion injection point 18 is avoided by the principle similar to that of the first embodiment.

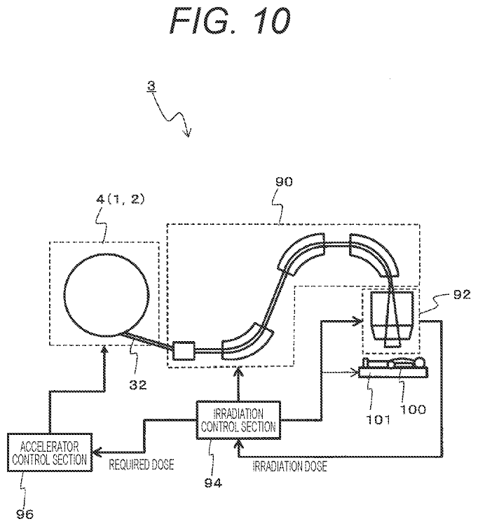

FIG. 10 illustrates a particle therapy system 3 according to an application embodiment of the present invention. The particle therapy system 3 is an apparatus using the eccentric trajectory type accelerator (1 or 2) according to the first embodiment or the second embodiment.

As illustrated in FIG. 10, the particle therapy system 3 irradiates a patient 100 with the energy of the charged particle beam as a value corresponding to a depth of a target volume from a body surface. The particle therapy system 3 includes an eccentric trajectory type accelerator 4, a beam transport device 90, an irradiation nozzle 92, a couch 101, an irradiation control section 94, and an accelerator control section 96. The irradiation control section 94 and the accelerator control section 96 may include a processor that executes processing to be described below by executing a program.

The eccentric trajectory type accelerator 4 is the eccentric trajectory type accelerator according to the first embodiment or the second embodiment. The eccentric trajectory type accelerator 4 accelerates the ions forming the charged particle beam. The beam transport device 90 transports the charged particle beam accelerated by the eccentric trajectory type accelerator 4 to the irradiation nozzle 92. The irradiation nozzle 92 irradiates the target volume in the patient 100 fixed to the couch 101 with the charged particle beam transported by the beam transport device 90. The irradiation nozzle 92 shapes the charged particle beam according to a shape of the target volume, and irradiates each irradiation spot in the target volume with the shaped charged particle beam.

The irradiation nozzle 92 includes a dose monitor, and measures an irradiation dose for each irradiation spot. Based on the measured value obtained in this manner, the irradiation control section 94 calculates a required dose for each irradiation spot. The irradiation control section 94 outputs the required dose for each irradiation spot to the accelerator control section 96. The accelerator control section 96 controls the energy, the extraction timing, and the like of the charged particle beam in the eccentric trajectory type accelerator 4 based on the required dose.

Note that, the beam transport device 90 of the particle therapy system 3 is not limited to a fixed beam transport device. The beam transport device 90 may be a transport system that is rotatable around the patient 100 together with the irradiation nozzle 92 called a rotating gantry. The number of irradiation nozzles 92 is not limited to one, and a plurality of irradiation nozzles may be provided. The form of the particle therapy system 3 may be a form in which the beam transport device 90 is not provided and the charged particle beam is directly transported from the eccentric trajectory type accelerator 4 to the irradiation nozzle 92.

### REFERENCE SIGNS LIST

1, 2, 4 eccentric trajectory type accelerator
3 particle therapy system
10 container
12 laser source
14 laser injection through-hole
16 laser beam
18 ion injection point
20 target
22 radiofrequency acceleration cavity
24 variable capacitor
25 DC power supply
26 acceleration radiofrequency power supply
28 conductive wire through-hole
30 beam through-hole
32 high energy beam transport device
34 motor
36 yoke
38 main coil
40 vacuuming through-hole
42 extraction septum magnet
44 kick magnetic field generating shim
46 disturbance electrode
48 dee electrode
52 acceleration gap
54 maximum energy trajectory
56 radiofrequency system through-hole
60 cryostat
61 ion passage region
62U, 62L magnetic pole
64 ion generator
66 housing
68 optical vacuum window
71 condenser lens
72 plasma
74 ion
80 optical mirror
90 beam transport device
92 irradiation nozzle
94 irradiation control section
96 accelerator control section
100 patient
101 couch

1. An accelerator, comprising:
a laser source; and
a target which emits ions by being irradiated with a laser beam emitted from the laser source,
wherein the ions emitted from the target are accelerated.

2. The accelerator according to claim 1, further comprising:
an acceleration electrode structure which accelerates the ions; and
a coil which generates a magnetic field in an ion passage region through which the ions pass,
wherein the target is provided at a position away from a center of a trajectory of the ion having a certain constant energy.

3. The accelerator according to claim 2, further comprising:
a container which has the ion passage region therein; and
a laser injection through-hole which extends from an outside of the container to an internal space of the container,
wherein the laser source irradiates the target with the laser beam via the laser injection through-hole.

4. The accelerator according to claim 1, further comprising:
a container which forms a columnar space therein;
an acceleration electrode structure which accelerates the ions in a circumferential direction of the columnar space; and
a coil which generates a magnetic field in an axial direction of the columnar space,
wherein the target is disposed at a position away from a central axis of the columnar space.

5. The accelerator according to claim 4, further comprising
a laser injection through-hole which extends from an outside of the container to an internal space of the container,
wherein the laser source irradiates the target with the laser beam via the laser injection through-hole.

6. The accelerator according to claim 1,
wherein the acceleration electrode structure further includes
a dee electrode which forms a spread-out cavity space, the dee electrode having a dee electrode opening spreading out in two different directions as viewed from a base side on which the spread-out cavity space spreads out, and
a dummy dee electrode which has an annular conductor facing an edge of the dee electrode opening, and
the target is disposed between the dee electrode and the dummy dee electrode.

7. The accelerator according to claim 6, further comprising:
a housing which is formed by using a conductor and accommodates the target, the housing being disposed between the dee electrode and the dummy electrode; and
a power supply which generates an electric field between the housing and the dee electrode,
wherein the housing has a window into which the laser beam is injected and an ion extraction hole from which the ions are extracted.

8. The accelerator according to claim 1, further comprising
a plurality of the targets made of different materials,
wherein a plurality of types of ions having different masses or valences which are emitted from the plurality of targets are accelerated.

9. A particle therapy system, comprising:
the accelerator according to claim 1;
a beam transport device which transports the ions extracted from the accelerator; and
an irradiation nozzle which irradiates a patient with the ions transported by the beam transport device.
